(19) United States
(12) Patent Application Publication
(10) Pub. No.: US 2024/0191313 A1
(43) Pub. Date: Jun. 13, 2024

(54) CDI Rapid Test For COVID-19 Variants of Concern

(71) Applicant: Hackensack Meridian Health, Inc., Edison, NJ (US)

(72) Inventors: Yanan Zhao, Livingston, NJ (US); Liang Chen, Livingston, NJ (US); David S. Perlin, New York, NY (US)

(73) Assignee: Hackensack Meridian Health, Inc., Edison, NJ (US)

(21) Appl. No.: 18/285,128

(22) PCT Filed: Apr. 6, 2022
(86) PCT No.: PCT/US2022/023670

§ 371 Date: Sep. 29, 2023

Related U.S. Application Data
(60) Provisional application No. 63/231,424, filed on Aug. 10, 2021.
(60) Provisional application No. 63/191,484, filed on May 21, 2021.
(60) Provisional application No. 63/171,377, filed on Apr. 6, 2021.

(51) Int. Cl. C12Q 1/70

(52) U.S. Cl. CPC C12Q 1/701; C12Q 2600/156

(57) ABSTRACT

A method, detection assay, and kit for rapidly detecting mutations of the SARS-COV-2 virus includes preparing a detection assay, performing an asymmetric real time-polymerase chain reaction (RT-PCR) on the detection assay using a Mic Real Time PCR cycler, and analyzing a melting curve to detect peaks at a 484 and a 501 codon of a gene, or detect peaks at a 452 codon and a 478 codon of a gene. The method, detection assay, and kit also provides for detecting of the SARS-COV-2 virus and mutations thereof. Disclosed is a high-throughput test that can detect multiple variants of the SARS-COV-2 virus within two-and-a-half hours, and is a major advance in tracking the virus and in treating patients. The test can detect UK, Brazil, South African, and Omicron variants, as well as others containing, for example, a key E484K mutation, which are gaining prominence as the virus evolves.

### CROSS-REFERENCE TO RELATED APPLICATION

The present application claims the benefit of the filing date of U.S. Provisional Patent Application No. 63/171,377, filed Apr. 6, 2021, and U.S. Provisional Patent Application No. 63/191,484, filed May 21, 2021, and U.S. Provisional Patent Application No. 63/231,424, filed Aug. 10, 2021, the disclosures of all are hereby incorporated herein by reference.

### TECHNICAL FIELD

The present application is related to a detection assay to identify mutations of the SARS-COV-2 virus. The detection assay is designed to identify principal mutations conferring amino acid changes at positions at least at E484, L452, and N501 of the spike protein.

### BACKGROUND OF RELATED ART

An outbreak of pneumonia caused by a novel coronavirus (SARS-COV-2) in Wuhan City, Hubei Province, China was initially reported to the World Health Organization (WHO) on Dec. 31, 2019. The emergence and rapid spread of SARS-COV-2 to numerous areas throughout the world has necessitated preparedness and response in laboratories, as well as health care and other areas of society in general. Since its initial discovery, the SARS-COV-2 virus has quickly adapted to various pressures from antiviral therapy and host immunity and evolved independently into several SARS-COV-2 variants of concern (VOC). These variants include the United Kingdom (UK) (B.1.1.7, a.k.a. 501Y.V1) variant, South Africa (SA) (B.1.351, a.k.a. 501Y.V2) variant, and Brazil (BZ) (P.1, a.k.a. 501Y.V3) variant.

These variants are concerning because they may acquire the ability to escape from neutralizing antibody responses to reduce vaccine efficacy or show increased transmissibility by making key mutations in the spike protein. Though each variant can cause several spike mutations, studies have found that resistance to neutralizing antibody is largely associated with the E484K mutation. Studies have also found that there is one common key mutation in all three VOC, the N501Y mutation. This mutation is considered to enhance the binding between spike and the ACE2 receptor in human cells, which contributes to increased transmissibility and possibly virulence. It has also been discovered that the L452R mutation is another mutation associated with the variants of the SARS-COV-2 virus, specifically for the Indian variant.

Mutation is expected to occur in virus evolution, and in many cases, does not produce devastating new viral characteristics. Therefore, it is not the number of variants nor the origin that is of concern to one of skill in the art, but it is the type of mutations the variants contain. Because there is confirmed association between E484K and N501Y mutations and their effect on neutralizing antibodies used in therapy and increasing virus transmissibility, there is a need to identify any variants carrying these two mutations. Further, because there is a similar effect as a result of L452R and E484Q mutations, there is a need to identify any variants carrying these mutations.

Still further, since the official designation of the newly emerged, highly transmissible SARS-COV-2 variant (B.1.617.2) (“the Delta variant”) as a variant of concern (“VOC”) by WHO, the Delta variant has become the predominant variant of SARS-COV-2 circulating globally. Among an array of mutations Delta variant carries, the presence of both L452R and T478K in the S protein is unique for Delta and can, therefore, be used as a diagnostic marker to differentiate Delta variant from other VOCs and variants of interest (VOIs).

The first Omicron (B.1.1.529 BA.1) variant was officially classified as VOC by WHO in Nov. 26, 2021. This variant has been detected at faster rates than previous surges in infection, suggesting that this variant may have a growth advantage. Omicron variant has more than 12 mutations in the spike proteins, of which E484A, N501Y and N505H are signatures to distinguish this variant from other VOCs.

Omicron subvariant BA.2 is a sublineage emerged from Omicron BA.1. Since Jan. 24, 2022, BA.2 has become dominant in almost all geographic areas globally. BA.1 and BA.2 share 32 mutations, but differ by 28 mutations. Of note, BA.2 does not have deletion (469-70) in the S protein, a major mutation found in BA.1. Therefore, 69-70WT is the signature of BA.2 following identification of Omicron variant.

Currently, whole genome sequencing (WGS) is being used as the main tool for epidemiological monitoring of SARS-COV-2 variants. However, there is a long turn-around-time for SARS-COV-2 WGS to get results, where it takes days if not weeks. Further, the demand on bioinformatic expertise for data analysis makes identification of concerning variants lagged far behind laboratory COVID diagnosis. Thus, there is an unmet need for faster and simpler diagnostics that can be used in a high-throughput fashion to increase the capacity of SARS-COV-2 variant detection in real-time.

### SUMMARY OF THE PRESENT INVENTION

The presently disclosed solves the issues of current state of the art, meets the above-mentioned requirements, and provides many more benefits. A method, detection assay, and kit for rapidly detecting mutations of the SARS-COV-2 virus, including preparing a detection assay, performing an asymmetric real time-polymerase chain reaction (RT-PCR) on the detection assay using a Mic Real Time PCR cycler, and analyzing a melting curve to detect peaks at a 484 and a 501 codon of the S gene. A detection assay is described for revealing mutations of the SARS-COV-2 virus, including a limiting primer represented by SEQ ID NO.1, an excess primer represented by SEQ ID NO.2, a first molecular beacon represented by SEQ ID NO. 3, and a second molecular beacon represented by SEQ ID NO. 4. The detection assay may also include a E484Q molecular beacon probe represented by SEQ ID NO. 5, a 452WT molecular probe represented by SEQ ID NO. 8, a limiting forward primer represented by SEQ ID NO. 6 and an excess reverse primer represented by SEQ ID NO. 7.

In another aspect, a method, detection assay, and kit for detecting mutations of the SARS-COV-2 Delta variant, includes preparing a detection assay, performing an asymmetric real time-polymerase chain reaction (RT-PCR) on the detection assay using a Mic Real Time PCR cycler, and analyzing a melting curve to detect peaks at a 452 codon and a 478 codon of the S gene. A detection assay is described for revealing mutations of the SARS-COV-2 Delta variant. including a limiting primer represented by SEQ ID NO.1, an excess primer represented by SEQ ID NO.2, and a molecular beacon represented by SEQ ID NO. 9.

The detection assay may also include a 452WT molecular probe represented by SEQ ID NO. 8, a limiting forward primer represented by SEQ ID NO. 6, and an excess reverse primer represented by SEQ ID NO. 7.

In another aspect, a method, detection assay, and kit for detecting mutations of the SARS-COV-2 Omicron variant, includes preparing a detection assay, performing an asymmetric real time-polymerase chain reaction (RT-PCR) on the detection assay using a Mic Real Time PCR cycler, and analyzing a melting curve to detect peaks at a 452, 484 and 501 codons of the S gene. A detection assay is described for revealing mutations of the SARS-COV-2 Omicron variant, including a limiting primer represented by SEQ ID NO.1, an excess primer represented by SEQ ID NO.2, and molecular beacons represented by SEQ ID NO. 3 and SEQ ID NO. 4.

In another aspect, following identification of Omicron variant, a method, detection assay, and kit for detecting mutations of the BA.2 subvariant, includes preparing a detection assay, performing an asymmetric real time-polymerase chain reaction (RT-PCR) on the detection assay using a Mic Real Time PCR cycler, and analyzing a melting curve to detect peaks at 69-70 codons of the S gene. A detection assay is described for revealing the lack of Δ69-70 in BA.2 subvariant, including a limiting primer represented by SEQ ID NO.12, an excess primer represented by SEQ ID NO.13, and molecular beacons represented by SEQ ID NO. 10 and SEQ ID NO. 11.

In still another aspect, disclosed herein is a novel molecular diagnostic assay disclosed herein capable of identifying signature mutations within 2.5 h from sample preparation to report and used to screen clinical samples such as those from nasopharyngeal swabs (NS). Variants, including Alpha (B.1.1.7, a.k.a. 501Y.V1), Beta (B.1.351, a.k.a. 501Y.V2), Gamma (P.1, a.k.a. 501Y.V3), Delta (B.1.617.2), and Omicron (B.1.1.529) variants, are concerning because they either resist neutralizing antibody and possibly reduce vaccine efficacy or show increased transmissibility, via making some key mutations in the spike protein.

The present novel genotyping is based in one embodiment on the thermal dynamic difference of molecular beacon (MB) binding with a perfectly complementary target or mismatch target. To generate single-stranded target DNA for the MB probe, an asymmetric reverse transcription (RT)-PCR assay was developed to amplify the mutation hotspot region covering both 484 and 501 codons of the S gene (supplemental material). Upon completion of thermal cycling, a melting curve analysis is performed to characterize dissociation between the single-stranded DNA product and two differentially labelled MB probes, to enable simultaneous genotyping at both loci. Owing to the probe design, the wildtype (WT) template is expected to generate a higher melting temperature (Tm) than that of the mutated genotype at a corresponding locus.

### BRIEF DESCRIPTION OF THE DRAWINGS

FIGS. 1A-1B are graphs representing the melting curves for WT and mutated genotypes at 484 and 501, respectively, detected from the detection assay of the present application.

FIG. 2 represents the prevalence of E484K and N501Y from late December 2020 to March 2021.

FIG. 3 represents the E484K and N501Y occurrence in different hospitals in February and March 2021.

FIG. 4 is an illustration of a phylogenetic tree of 74 SARS-COV-2 genomes.

FIGS. 5A-5B are graphs representing the melting curves for WT and mutated genotypes at 452 and 478, respectively, detected from a detection assay of the present application.

### DETAILED DESCRIPTION

Spike protein mutations E484K and N501Y carried by SARS-COV-2 variants have been associated with concerning changes of the virus, including resistance to neutralizing antibodies and increased transmissibility. While the concerning variants are fast spreading in various geographical areas, identification and monitoring of these variants are lagging far behind, due in large part to the slow speed and insufficient capacity of viral sequencing. In response to the unmet need for a fast and efficient screening tool, developed and disclosed herein are a single-tube duplex molecular assay for rapid and simultaneous identification of E484K and N501Y mutations from for example nasopharyngeal swab (NS) samples within 2.5 h from sample preparation to report. Using this tool, it is possible to screen thousands of clinical NS samples collected from COVID patients at multiple locations. Data revealed herein shows dramatic increases in the frequencies of both E484K and N501Y over time.

For purposes of this disclosure, the term “sample” as used herein refers to any sample that is taken from a subject (e.g., a human, such as a person suspect of infection) and contains one or more nucleic acids of interest. The term “nucleic acid” as used herein refers to a total nucleic acid including both deoxyribonucleic acid (DNA) and ribonucleic acid (RNA). The term “reaction” as used herein refers to any process involving a chemical, enzymatic or physical action that is indicative of the presence or absence of a nucleic acid of interest. An example of a “reaction” is an amplification reaction such as a polymerase chain reaction (PCR). The term “well” as used herein refers to a reaction at a predetermined location within a confined structure, e.g., a well-shaped vial, cell, or chamber in a PCR array.

As used herein, the term “detection assay” refers to a standardized procedure to detect the presence or absence of a particular nucleic acid of interest. The term “specimen” as used herein is obtained from nasal wash, aspirate, or a swab in a universal or viral transport media from a subject to be used in the sample.

The detection assay of the present application is an asymmetric real-time transcription polymerase chain reaction (RT-PCR) assay. The detection assay is a novel genotyping based on the thermal dynamic difference of molecular beacon binding with a perfectly complementary target or a mismatch target. The detection assay of the present application may provide results within 2.5 hours from sample preparation to report. Therefore, the detection assay of the present application is ideal to serve as a screening tool to facilitate downstream on-target WGS and to provide more efficient epidemiological surveillance.

The detection assay of the present application also includes performing a molecular beacon (MB) probe-based melting curve analysis. Melting curve analysis is useful in the study of various substances. In particular, nucleic acids have been studied extensively through melting curves, where differences in melting curves can be indicative of different nucleic acid sequences. Thus, the detection assay uses an asymmetric RT-PCR in conjunction with MB probe-based melting curve analysis.

This process is described in Rapid Detection of FKS-Associated Echinocandin Resistance in Candida glabrata. (Zhao Y, Nagasaki Y, Kordalewska M, et al. Rapid Detection of FKS-Associated Echinocandin Resistance in Candida glabrata. Antimicrobial agents and chemotherapy. 2016 November; 60(11):6573-6577. doi: 10.1128/aac.01574-16. PubMed PMID: 27550360; PubMed Central PMCID: PMCPMC5075061. eng.) During asymmetric RT-PCR, a single stranded amplicon is produced, which allows the probe to anneal and generate fluorescence at low temperature.

The fluorescence intensity decreases when the probe slowly dissociates from the target as a result of gradually increased temperature in the subsequent melting analysis. The temperature melting temperature, Tₘ, is determined based on the curve plotted by the fluorescence intensity change as a function of temperature. The Tₘ value varies when the target changes from perfectly matched sequence to mismatched sequence in the testing system, providing a solid basis for wild-type (WT)/non-WT discrimination.

The detection assay of the present application produces a single-stranded amplicon from the mutation hotspot region covering both 484 and 501 codons of the S gene. To generate single-stranded target DNA for the MB probe, the asymmetric RT-PCR assay was developed to amplify the mutation hotspot region covering both 484 and 501 codons of the S gene.

Upon completion of thermal cycling, a MB probe-based melting curve analysis is performed to characterize dissociation between the single-stranded DNA product and two differentially labeled molecular beacon probes, to enable simultaneous genotyping at both loci. The MB probes are prepared to detect the codons of the S gene that could detect the variants of COVID-19.

The MB probes are designed to contain the wildtype (WT) nucleic acid sequences comprising 69-70, 478, 484, and 501 amino acids. The 69-70dd-MB probe is designed to contain sequences comprising the 69-70 deletions. Based on the thermodynamic features of the molecular beacon, total energy needed to dissociate the perfectly complementary probe-target hybrid is greater than that need to dissociate the mismatched probe-target hybrid. Thus, a higher Tₘ is generated for each probe in the presence of the single-stranded product carrying the WT target sequence, compared to the Tₘ obtained in the presence of the single-stranded product harboring mutations in the probe binding region. The 478WT-MB probe is represented by SEQ ID NO. 9. the 484WT-MB probe is represented by SEQ ID NO. 3, and the 501WT-MB probe is represented by SEQ ID NO. 4. An additional MB probe to detect E484Q mutation has been developed and is represented by SEQ ID NO. 5. The E484Q MB probe is readily used simultaneously with the 484WT-MB and 501WT-MB in the same assay.

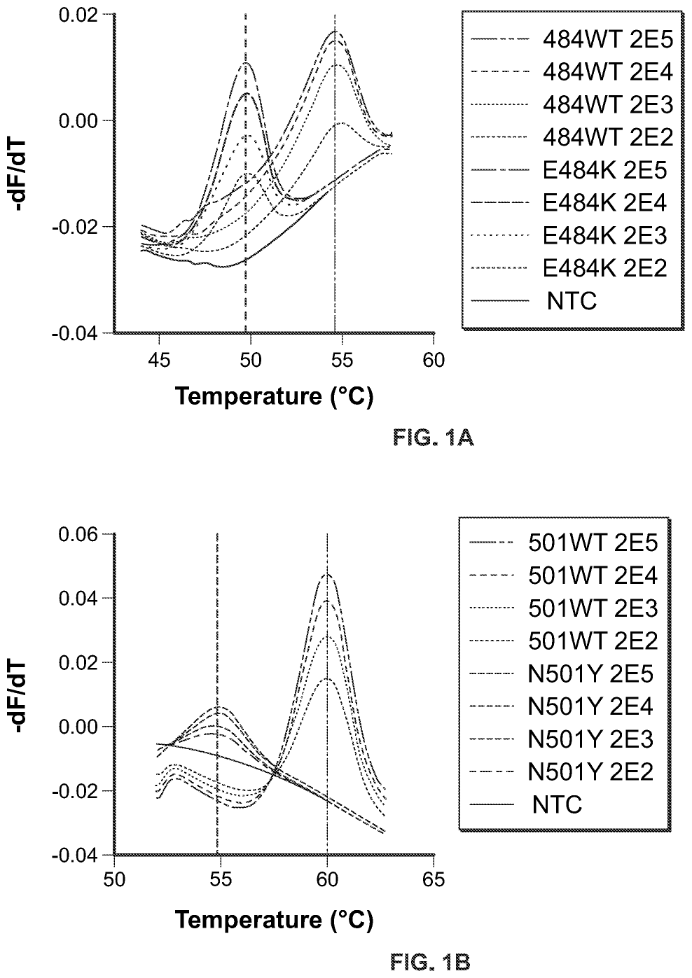

In FIGS. 1A-1B, E484 WT is featured for a Tₘ at 54.85° C.±0.19° C., ˜5° C. higher than the Tₘ of E484K (49.81° C.±0.07° C.). Similarly, the signature Tₘ for N501 WT was 59.97° C.±0.09° C., which is higher than 54.78° C.±0.12° C. for N501Y.

Further shown in FIG. 1 are melting profiles for E484WT and E484K (left panel FIG. 1A), and those for N501WT and N501Y (right panel FIG. 1B). For each genotype, 10-fild serial RNA dilutions containing 2×10⁵-200 genome equivalents/reaction were tested Dashed lines indicate the Tm value of corresponding genotype. NTC is the abbreviation for no template control. To confirm the genotyped RNA samples, six different reference viral strains were tested in a blinded fashion. From the blind test, the detection assay correctly genotyped RNA samples extracted from the six different reference viral strains, including one WT (SARS-COV-2 USA WA1/2020), two B.1.1.7 variants (SARS-COV-2 hCoV-19/USA/CA_CDC_5574/2020 and SARS-COV-2 hCoV-19/England/204820464/2020), and two B.1.351 variants (SARS-COV-2 hCoV-19/South Africa/KRISP-EC-K005321/2020 and SARS-COV-2 hCoV-19/South Africa/KRISP-K005325/2020) purchased from BEI resources, and one E484K variant isolate recently obtained from our network hospital.

The analytical sensitivity of the assay was evaluated against 10-fold serial dilutions of RNA prepared from each of the reference viral strain. The assay can reliably identify as low as 200 copies of 484WT, 200 copies of E484K, 20 copies of 501WT, and 200 copies of N501Y per reaction, respectively.

In another embodiment, the detection assay of the present application produces a single-stranded amplicon from the mutation hotspot region covering 452 codon of the S gene. To generate single-stranded target DNA for the MB probe, the asymmetric RT-PCR assay was developed to amplify the mutation hotspot region covering the 452 codon of the S gene, where the primers are represented by SEQ ID NOS. 6 and 7. Upon completion of thermal cycling, a MB probe-based melting curve analysis is performed to characterize dissociation between the single-stranded DNA product and the 452WT molecular beacon probe, to enable genotyping at L452.

The MB probe is prepared to detect the codons of the S gene that could detect the variants of COVID-19. An 452WT MB probe has been developed to detect the L452R mutation and is represented by SEQ ID NOs. 8.

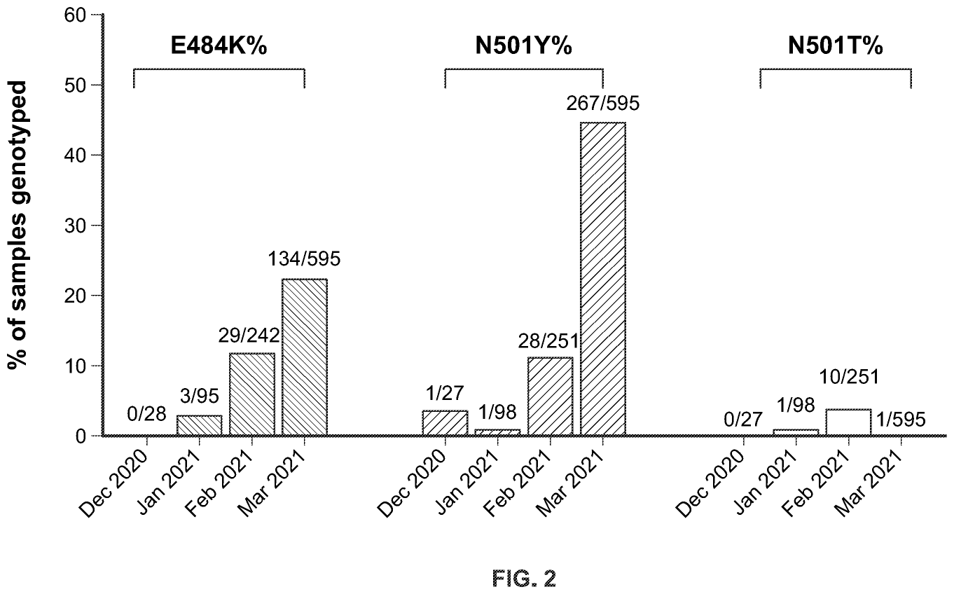

FIG. 2 illustrates the increasing prevalence of E484K and N501Y from late December 2020 to March 2021 supporting the need for a detection assay that can detect the mutations of the COVID-19 virus.

The upper respiratory specimens to be used in samples for the detection assay may be obtained from nasal wash, aspirate, or a swab in a universal or viral transport media. In particular, the upper respiratory specimens may be obtained from, but is not limited to, a Nasopharyngeal wash/aspirate, Nasal aspirate, Nasopharyngeal swab, Oropharyngeal swab, Anterior nasal swab, Mid-turbinate nasal swab, or tracheal aspirate. Other specimens such as saliva may also be considered as suitable sources for detection of virus.

To use the detection assay, an asymmetric RT-PCR was carried out on a Mic Real Time PCR Cycler in a 20 μl reaction volume using the One Step PrimeScript™ RT-PCR Kit (Perfect Real Time) (Takara). This duplex assay contained 10 μl of one step RT-PCR Buffer III, 0.4 μl of PrimeScript RT enzyme Mix II, 0.4 μl of TakaRa Ex Taq HS (5 U/μl), 40 nM of the forward primer of SEQ ID NO. 1, 1 μM of the limiting primer of SEQ ID NO. 2 (10 μM), 100 μM of both molecular beacons, 484WT-MB and 501WT-MB, represented of SEQ ID NOS. 3 and 4, and alternatively SEQ ID NOS. 10 and 11, and 5 μl of RNA or heat-inactivated template.

The thermal cycling profile may be about 42° C. for 5 min for reverse transcription, followed by 95° C. for 10 sec then 50 cycles of 95° C. for 5 sec and 60° C. for 20 sec. Immediately after amplification. MB probe-based melting curve analysis is initiated as a minute incubation at 95° C., after which the sample was melted from 47.5° C. to 58.5° C. with a ramp rate of 0.1° C./s for the 484WT-MB and melted from 53° C. to 63° C. with a ramp rate of 0.1° C./s for the 501WT-MB.

The use of the detection assay will be further described in the following examples.

### Clinical Validation of Melting Temperatures

Nasopharyngeal swab samples (“samples”) were collected from COVID patients. A total of 1135 samples collected between late December 2020 and March 2021 from 8 HMH hospitals with a cycle of threshold (Ct) value<37 in SARS-COV-2 N2 RT-PCR test were then tested for spike mutation screening.

The screening procedure was speed up by using an extraction-free sample process method. In the extraction-free sample process method, a 50 μl aliquot of sample is heat inactivated at 95° C. for 5 minutes, prior to genotyping test. From this, 960 and 971 samples yielded identifiable signals for the 484 probe and the 501 probe, respectively.

The proportion of E484K was 17.2% ( 165/960), and it was 30.6% ( 297/971) for N501Y. There were 6 samples carrying both E484K and N501Y, and remaining samples flagged as mutants only carry one of the two signature mutations. In addition, we discovered a new genotype at the 501-probe binding site from 12 samples ( 12/971, 1.2%), which thereafter was confirmed to be a N501T (AAT>ACT) mutation in subsequent sequencing. The melting profile of N501T is markedly different from that of WT and N501Y, with a signature Tm of 56.41° C.±0.15° C.

Also captured was one sample eliciting a distinct 484 Tm at 48.88° C. ˜1° C. lower than E484K and ˜6° C. lower than 484WT. Amplicon sequencing verified that this sample carries an E484Q (GAA>CAA) mutation. To confirm the screening results, we performed whole genome sequencing with a panel of 74 samples representing different genotypes flagged by this screening tool, including 24 E484K, 25 N501Y, 5 N501T, and 20 WT at 484 and 501 loci. Within this WGS confirmed panel, our assay achieved 100% sensitivity and specificity for both 484 and 501 genotyping.

### Whole Genome Sequencing

Whole genome sequencing (WGS) was performed with a panel of 74 samples representing different genotypes flagged by this screening tool, including 24 E484K, 25 N501Y, 5 N501T, and 20 WT at 484 and 501 loci. Indeed, WGS results are 100% consistent with our genotyping determination. Genomic analysis showed that the majority of the E484K cases (n=19) fell within the B.1.526 lineage, a recent clone emerged from New York, and the rest belong to clade 20C B.1 lineage (n=2), and clade 20B under R.1 (n=2) and B.1.1.309 lineage (n=1), respectively. All N501Y cases except one are members of B.1.1.7 lineage.

The present disclosure, among other things, demonstrates a novel and easy molecular diagnostic assay may be used as a convenient tool for large scale of SARS-COV-19 variant screening, thus, to enable highly efficient epidemiological monitoring. The detection assay is highly accurate and sensitive to new mutations within the probe binding site. Because the virus is continuously evolving, new mutations within the probe binding site may generate melting profiles similar to one of the target mutations tested, if the mutation causes thermal dynamic change close to one of those tested, which comprises the diagnostic performance for defined signature mutation. However, because of the nature of the assay design, any mutation potentially occurring within the probe binding region would result in Tₘ shift from that of the WT. This feature ensures that the assay has the capability of discriminating mutations for the WT.

The detection assay may be expanded by adding additional probes as new mutations of the SARS-COV-19 virus are discovered or learned of.

### Viral Culture and RNA Extraction

Viral culture stocks of SARS-COV-2 USA WA1/2020 (WT), 20 SARS-COV-2 hCoV-19/USA/CA_CDC_5574/2020 (B.1.1.7), SARS-COV-2 hCoV-19/England/204820464/2020 (B.1.1.7), SARS-COV-2 hCoV-19/South Africa/KRISP-EC-K005321/2020 (B.1.351), and SARS-COV-2 hCoV-19/South Africa/KRISP-K005325/2020 (B.1.351) were obtained from BEI Resources, NIAID (Manassas, VA). One mutant carrying E4848K but not N501Y was recently isolated and obtained from a HMH network hospital. All strains were propagated on VeroE6/TMPRSS2 cell line (SEKISUI XenoTech, KS) in a Biosafety level (BSL) laboratory. Supernatant of the viral culture was proteinase K treated (200 μg/ml) and heat inactivated at 95° C. for 10 min prior to RNA isolation in the BSL-2 laboratory using QIAamp viral RNA mini kit (Qiagen, Germantown, MD, USA).

### Primer and Probe Design

One set of primers were designed to amplify a 148-nt region of the SARS-COV-2 genomic RNA covering E484 and N501 of the spike protein. Forward primer SEQ ID NO. 1: S484F (5′-GAGAGAGATATTTCAACTGAAATCTATCAGGC-3′) was used as limiting primer, and SEQ ID NO. 2: S501R (5′-AAAGTACTACTACTCTGTATGGTTGGT-3′) was used as excess primer. Two differentially labeled molecular beacons, SEQ ID NO. 3: 484WT-MB (5′-FAM-CGTGACATGGTGTTGAAGGTTTTAATTGGTCACG-Dabcyl-3′) and SEQ ID NO. 4: 501WT-MB (5′-Quasar670-CGCGACACCCACTAATGGTGTTGGTTACCGTCGCG-BHQ2-3′) (underlining signifies the stem portion of the molecular beacon), were designed to contain the WT sequences of E484 and N501, respectively. Depending on the implementation, alternatively SEQ ID NOs. 10-11 may be utilized in place of SEQ ID NOs. 3-4, respectively.

### Asymmetric RT-PCR and Melting Curve Analysis

Asymmetric RT-PCR was carried out on the Mic Real Time PCR Cycler (Bio Molecular Systems, software micPCRv2.8.13) in a 20 μl reaction volume using the One Step PrimeScript™ RT-PCR Kit (Perfect Real Time) (Takara, Mountain View, CA, USA). This duplex assay contained 10 μl of one step RT-PCR Buffer III, 0.4 μl of PrimeScript RT enzyme Mix II, 0.4 μl of TakaRa Ex Taq HS (5 U/μl), 40 nM of S484F, 1 μM of S501R (10 μM), 100 μM of both 484WT-MB and 501WT-MB, and 5 μl of RNA or heat-inactivated template. Thermal cycling profile was 42° C. for 5 min for reverse transcription, followed by 95° C. for 10 sec then 50 cycles of 95° C. for 5 sec and 60° C. for 20 sec. Immediately after amplification, melting curve analysis was initiated as a minute incubation at 95° C., after which it was melted from 47.5° C. to 58.5° C. with a ramp rate of 0.1° C./s for the 484WT-MB and melted from 53° C. to 63° C. with a ramp rate of 0.1° C./s for the 501WT probe.

### Viral Genome Sequencing and Analysis

Viral RNA from swabs was extracted using QIAcube Connect (Qiagen), following the manufacturer's instructions. SARS-COV-2 targeted assay libraries were prepared using the QIAseq SARS-COV-2 Primer Panel and cDNA Synthesis for Illumina kits (Illumina, San Diego, CA. USA). Adapter sequences and low quality (Q<20) bases were trimmed from the raw reads, using Cutadapt v2.101 (https://github.com/marcelm/cutadapt/). Processed reads were then mapped to the SARS-COV-2 genome reference using BWA-MEM v0.7.172 and genome sequences were determined by Samtools v1.11 and bcftools v1.11. The genome clades and lineages were determined by Nextclade server and Pangolin v2.3.0, respectively. Genomes were aligned using nextalign v0.2.0, and a maximum likelihood phylogenetic tree was constructed using IQ-TREE v2.1.2 with automatic model selection and 1000-bootstrap replicates. The tree was annotated using iTOL v6.0.

### Clinical Screening

Clinical samples included in this study were de-identified nasopharyngeal swabs obtained from 8 different sites within HMH network and banked in the biorepository of our center. All samples were collected in standard viral transport media and stored at −80° C. upon receipt. An extraction-free method was used to process samples prior to genotyping analysis. Briefly, a 50 μl of aliquot was taken from each swab and mixed with 6.5 μl of proteinase K (20 mg/ml, Roche, Indianapolis, IN, USA), followed by heating up the mixture at 95° C. for 5 min. Then 5 μl of the processed sample was used directly as template for genotyping assay. Information of sample source and collection timeline was provided by HMH bio-R working group.

### Statistical Analysis

Melting temperatures or Tₘ values for E484 and N501 genotype were determined by melting curve analysis using the Mic Real-Time PCR software (micPCRv2.8.13). The melting curve and epidemiological distribution of variants were plotted and analyzed in GraphPad Prism version 9.0.0. We used χ² test or Fisher's exact test to compare the distribution between different locations. A P value less than 0.05 was considered statistically significant.

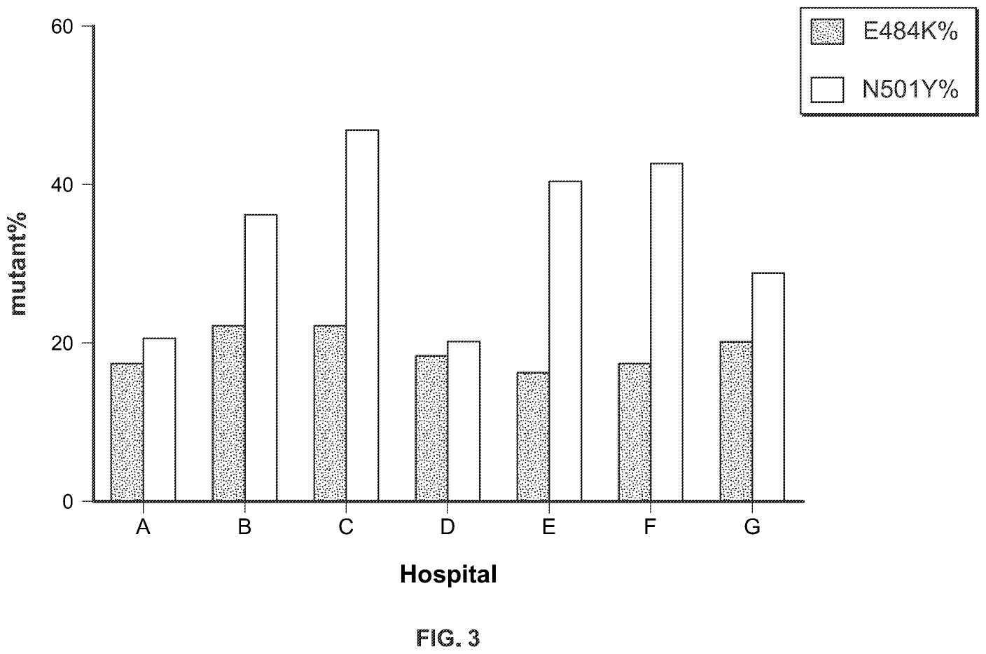

FIG. 3 illustrates the occurrence of E484K and N501Y in different hospitals in February and March 2021. In FIG. 3, seven hospitals, coded from A to G, were included in the analysis. Hospital H had only one sample (WT for both 484 and 501 loci) collected in January contributed to this study, therefore excluded from this analysis. While E484K occurrence was similar between different hospitals (χ²=3.61, P=0.73), N501Y distribution between hospitals was quite different (χ²=25.7, P=0.0003), ranging from 20.2% to 47.1% of the total number of genotyped samples in each hospital.

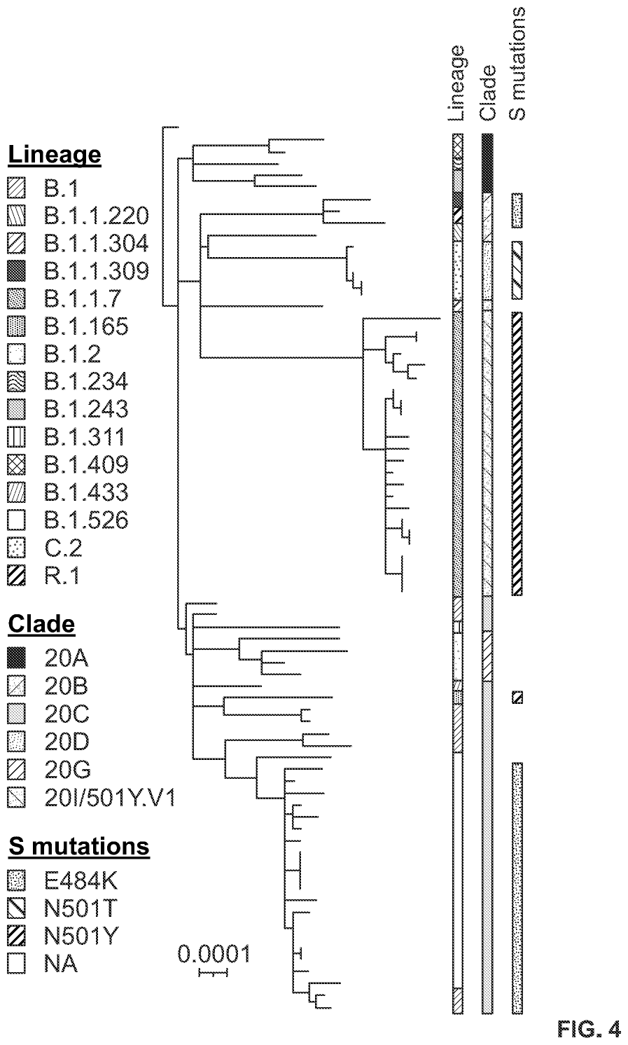

FIG. 4 is a maximum-likelihood phylogenetic tree of 74 SARS-COV-2 genomes from HMH network. The tree is rooted with the Wuhan/Hu-1 SARS-COV-2 reference (NC_045512.2) sequence and annotated using iTOL (www.itol.embl.de). The scale bar represents 0.0001 nucleotide substitutions per site. The SARS-COV-2 Pangolin lineage, NextStrain clade, S protein 484 and 501 mutations were illustrated by different color bars on the right.

### Delta Variant of SARS-COV-2

To use the detection assay to detect the Delta variant, both 452 and 478 assays were carried out on a Mic Real Time PCR Cycler in a 20 μl reaction volume using the One Step PrimeScript RT-PCR Kit (Perfect Real Time) (Takara). The 452 assay contained 10 μl of one step RT-PCR Buffer III, 0.4 μl of PrimeScript RT enzyme Mix II, 0.4 μl of Takara Ex Taq HS (5 U/μl), 40 nM of the forward primer S452F (SEQ ID NO.6), 1 μM of the reverse primer S452R (SEQ ID No.7), 100 μM of 452WT MB probe (SEQ ID NO.8), and 5 μl of RNA or heat-inactivated swab sample. The thermal cycling profile was 42° C. for 5 min for reverse transcription, followed by 95° C. for 10 sec then 50 cycles of 95° C. for 5 sec and 60° C. for 20 sec.

The 478 assay setup was the same as the 452 assay, except for the primers and MB probe. Specifically, the 478 assay used 40 nM of S484F (SEQ ID NO. 1), 1 μM of S501R (SEQ ID NO. 2), and 100 μM of 478WT MB probe (SEQ ID NO.9). Immediately after amplification, melting curve analysis was initiated as a minute incubation at 95° C., after which it was melted from 48° C. to 58° C. with a ramp rate of 0.1° C./s for the 452 assay, and melted from 52° C. to 65° C. with a ramp rate of 0.1° C./s for the 478 assay.

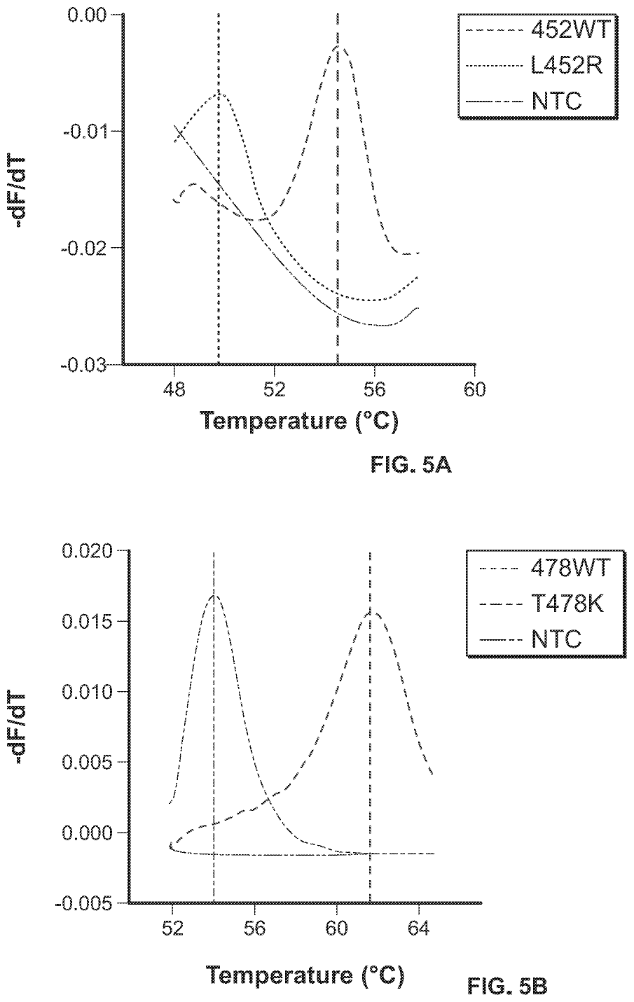

As shown in FIGS. 5A-5B, in the 452 assay, viral RNA harboring 452WT is featured for a Tm at 54.53° C.±0.17° C. ˜5° C. higher than the Tm of L452R (49.61° C.±0.14° C.). Similarly, viral RNA carrying 478WT has a signature Tm at 61.44° C.±0.23° C., and that carrying T478K is featured for a Tm at 54.05° C.±0.04° C.

The analytical sensitivity of each assay was evaluated against 10-fold serial dilutions of RNA prepared from the reference viral strains purchased from BEI resources, including one Delta strain (NR-55611) and one WT strain (SARS-COV-2 USA WA1/2020). The 452 assay can reliably identify as low as 200 copies of 452WT and 250 copies of L452R per reaction. The limit of detection for the 478 assay is 20 copies of 478WT and 50 copies of T478K pre reaction, respectively.

The 452 and 478 assays may be used to screen Delta variants from nasopharyngeal swab samples collected from COVID patients. All samples with a Ct value<39 in SARS-COV-2 N2 RT-PCR test are subjected to the variant screening. Thus far, a total of 119 samples from months of June and July in 2021 were screened for 452 and 478 genotyping, and the positivity rate for the presence of both L452R and T478K was 70% ( 19/27) in June 2021 and 90% ( 83/92) in July 2021.

### Omicron Variant of SARS-COV-2

To use the detection assay to detect the Omicron variant, both 452 and 484-501 assays were carried out on a Mic Real Time PCR Cycler in a 20 μl reaction volume using the One Step PrimeScript RT-PCR Kit (Perfect Real Time) (Takara). The 452 assay is the same as description [00058], and the 484-501 assay is the same as description [00052].

To use the detection assay to identify BA.2 subvariant following Omicron detection, the 69-70 assay may be carried out on a Mic Real Time PCR Cycler in a 20 μl reaction volume using the One Step PrimeScript RT-PCR Kit (Perfect Real Time) (Takara). This assay contained 10 μl of one step RT-PCR Buffer III, 0.4 μl of PrimeScript RT enzyme Mix II, 0.4 μl of Takara Ex Taq HS (5 U/μl), 40 nM of the forward primer 69-70F (SEQ ID NO.12), 1 μM of the reverse primer 69-70R (SEQ ID No.13), 100 μM of 69-70WT-MB probe (SEQ ID NO.10), 100 μM of 69-70dd-MB probe (SEQ ID NO.11) and 5 μl of RNA or heat-inactivated swab sample. The thermal cycling profile was 42° C. for 5 min for reverse transcription, followed by 95° C. for 10 sec then 50 cycles of 95° C. for 5 sec and 60° C. for 20 sec.

Illustrated below and herein are exemplary sequence listings. Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

SEQUENCE LISTING

SEQ ID NO. 1: S484F
5′-GAGAGAGATATTTCAACTGAAATCTATCAGGC-3′
SEQ ID NO. 2: S501R
5′-AAAGTACTACTACTCTGTATGGTTGGT-3′
SEQ ID NO. 3: 484WT-MB probe
5′-FAM-CGTGACATGGTGTTGAAGGTTTTAATTGGTCACG-
Dabcyl-3′
SEQ ID NO. 4: 501WT-MB probe
5′-Quasar670-CGCGACACCCACTAATGGTGTTGGTTACCGTCGCG-
BHQ2-3′
SEQ ID NO. 5: E484Q-MB probe
5′-CR610-CGTGACAATGGTGTTCAAGGTTTTAATTGGTCACG-
BHQ2-3′
SEQ ID NO. 6: S452F
5′-TTTTACAGGCTGCGTTATAGCTTGGA-3′
SEQ ID NO. 7: S452R
5′-CAGTTGAAATATCTCTCTCAAAAGG-3′
SEQ ID NO. 8: 452WT-MB probe
5′-HEX-CGCGACATTATAATTACCTGTATAGATTGTTTAGGGTCGCG-
DABCYL-3′
SEQ ID NO. 9: 478WT-MB probe
5′-CR610-CGACGCCGGTAGCACACCTTGTAATCGTCG-BHQ2-3′
SEQ ID NO. 10: 69-70WT-MB
5′-FAM-CGCGATATGCTATACATGTCTCTGGGATCGCG-Dabcyl-3′
SEQ ID NO. 11: 69-70dd-MB
5′-HEX-CGCGATTGCTATCTCTGGGACCAATGATCGCG-Dabcyl-3′
SEQ ID NO. 12: 69-70F
5′-CAGGACTTGTTCTTACCTTTCT-3′
SEQ ID NO. 13: 69-70R
5′-CAGGGTTATCAAACCTCTTAG-3′

What is claimed is:

1. A method for rapidly detecting a SARS-COV-2 virus and mutations thereof, comprising:
preparing a detection assay including preparing at least one molecular beacon (MB) probe for detecting a SARS-COV-2 virus or mutations thereof;
performing an asymmetric real time-polymerase chain reaction (RT-PCR) on the detection assay using a Mic Real Time PCR cycler;
analyzing a melting curve to detect peaks at a 484 and a 501 codon of a S gene, or detect peaks at a 452 and a 478 codon of the gene; and
detecting codons of the S gene to detect the virus or variants of the virus.

2. The method of claim 1, further comprises revealing mutations of the SARS-COV-2 virus by including a limiting primer represented by SEQ ID NO.1 or SEQ ID NO.12, an excess primer represented by SEQ ID NO.2 or SEQ ID NO.13, a first molecular beacon represented by SEQ ID NO. 3 or SEQ ID NO.10, and a second molecular beacon represented by SEQ ID NO. 4 or SEQ ID NO.11.

3. The method of claim 2, wherein when the limiting primer is represented by SEQ ID NO.12, the excess primer is represented by SEQ ID NO.13, and the first and the second molecular beacons are represented by SEQ ID NO. 10 and SEQ ID NO. 11, respectively, the detection assay reveals a lack of Δ69-70 in BA.2 subvariant; and
wherein when the limiting primer is represented by SEQ ID NO.1, the excess primer is represented by SEQ ID NO. 2, and the first and the second molecular beacons are represented by SEQ ID NO. 3 and SEQ ID NO. 4, respectively, the detection assay further comprises utilizing a E484Q molecular beacon probe represented by SEQ ID NO. 5, a 452WT molecular probe represented by SEQ ID NO. 8, a limiting forward primer represented by SEQ ID NO. 6, and an excess reverse primer represented by SEQ ID NO. 7.

4. The method of claim 1, wherein the analyzing the melting curve further comprises determining a thermal dynamic difference of the molecular beacon (MB) binding with a complementary target or a mismatch target.

5. The method of claim 4, further comprises characterizing dissociation between a single-stranded DNA product and two differentially labelled MB probes to enable simultaneous genotyping at both loci either at peaks of the 484 and the 501 codon of the gene, or peaks at the 452 and the 478 codon of the gene.

6. The method of claim 5, wherein the characterizing dissociation comprises generating a higher melting temperature (Tm) than that of a mutated genotype at a corresponding locus.

7. The method of claim 1, further comprises generating a single-stranded target DNA for the MB probe, and amplifying a mutation hotspot region covering both the 484 and the 501 codons, or both the 452 and the 478 codons of the S gene using an asymmetric reverse transcription (RT)-PCR assay.

8. A detection assay for detecting mutations of the SARS-COV-2 virus, comprising:
a limiting primer represented by SEQ ID NO.1 or SEQ ID NO.12;
an excess primer represented by SEQ ID NO.2 or SEQ ID NO.13;
a first molecular beacon (MB) probe;
a second molecular beacon (MB) probe; and
wherein the first and the second MB probes are designed to contain the wildtype (WT) nucleic acid sequences comprising a 452, a 478, a 484, or a 501 amino acids or codons of a S gene;
and the first and the second MB probes are prepared to detect the codons of the S gene that detect variants of a SARS-COV-2 virus, or reveal lack of Δ69-70 in BA.2 subvariant.

9. The detection assay of claim 8, wherein the first molecular beacon (MB) probe is represented by SEQ ID NO. 3 or SEQ ID NO.10, and the second molecular beacon (MB) probe is represented by SEQ ID NO. 4 or SEQ ID NO.11.

10. The detection assay of claim 8, further comprising:
a first assay comprising the limiting primer represented by SEQ ID NO.1, the excess primer represented by SEQ ID NO.2, and the first molecular beacon (MB) probe represented by SEQ ID NO. 8; and
a second assay comprising a second limiting primer represented by SEQ ID NO.6, a second excess primer represented by SEQ ID NO.7, and the second molecular beacon (MB) probe represented by SEQ ID NO. 9.

11. The detection assay of claim 8, wherein based on thermodynamic features of the first and the second molecular beacon (MB) probes total energy to dissociate a perfectly complementary probe-target hybrid is greater than that to dissociate a mismatched probe-target hybrid.

12. The detection assay of claim 11, wherein a higher melting temperature (Tₘ) is generated for each the first MB probe and the second MB probe in presence of a single-stranded product carrying a wild-type (WT) target sequence compared to the melting temperature (Tₘ) obtained in presence of the single-stranded product harboring mutations in a probe binding region.

13. The detection assay of claim 8, wherein a 478WT-MB probe is represented by SEQ ID NO. 9, a 484WT-MB probe is represented by SEQ ID NO. 3, and a 501WT-MB probe is represented by SEQ ID NO. 4.

14. The detection assay of claim 13, wherein a E484Q mutation is detected using either the first or the second MB probes that is a MB specific E484Q probe represented by SEQ ID NO. 5; and the MB specific E484Q probe is readily used simultaneously with the 484WT-MB probe and 501WT-MB probe in the same assay.

15. The detection assay of claim 8, wherein the 452 codon of the S gene has primers that are represented by SEQ ID NOS. 6 and 7; and the first or the second MB probes are a 452WT MB probe represented by SEQ ID NO. 8 for detecting a L452R mutation.

16. A kit for detecting SARS-COV-2, comprising:
a detection assay for revealing mutations of the SARS-COV-2 virus in a specimen;
wherein the detection assay includes a limiting primer, an excess primer, and a molecular beacon (MB) probe.

17. The kit of claim 16, wherein the molecular beacon (MB) probe is selected from a group consisting of SEQ ID NO. 3, SEQ ID NO. 4, SEQ ID NO. 5, SEQ ID NO. 8, SEQ ID NO. 9, SEQ ID NO. 10, SEQ ID NO. 11, and any combination thereof.

18. The kit of claim 17, wherein the detection assay further includes a E484Q molecular beacon (MB) probe represented by SEQ ID NO. 5, and a 452WT molecular beacon (MB) probe represented by SEQ ID NO. 8.

19. The kit of claim 16, wherein the limiting primer is either SEQ ID NO. 1 or SEQ ID NO. 12, and the excess primer is either SEQ ID NO. 2 or SEQ ID NO. 13.

20. The kit of claim 19, wherein the detection assay includes a 452WT molecular probe represented by SEQ ID NO. 8 and a 478WT molecular probe represented by SEQ ID NO. 9.


SEQUENCE LISTING (13 sequences)

SEQ ID NO: 1 - DNA, 32 bp, Artificial Sequence
  S484F
gagagagatatttcaactgaaatctatcaggc

SEQ ID NO: 2 - DNA, 27 bp, Artificial Sequence
  S501R
aaagtactactactctgtatggttggt

SEQ ID NO: 3 - DNA, 34 bp, Artificial Sequence
  484WT-MB probe
cgtgacatggtgttgaaggttttaattggtcacg

SEQ ID NO: 4 - DNA, 35 bp, Artificial Sequence
  501WT-MB probe
cgcgacacccactaatggtgttggttaccgtcgcg

SEQ ID NO: 5 - DNA, 35 bp, Artificial Sequence
  E484Q-MB probe
cgtgacaatggtgttcaaggttttaattggtcacg

SEQ ID NO: 6 - DNA, 26 bp, Artificial Sequence
  S452F
ttttacaggctgcgttatagcttgga

SEQ ID NO: 7 - DNA, 25 bp, Artificial Sequence
  S452R
cagttgaaatatctctctcaaaagg

SEQ ID NO: 8 - DNA, 41 bp, Artificial Sequence
  452WT-MB probe
cgcgacattataattacctgtatagattgtttagggtcgcg

SEQ ID NO: 9 - DNA, 30 bp, Artificial Sequence
  478WT-MB probe
cgacgccggtagcacaccttgtaatcgtcg

SEQ ID NO: 10 - DNA, 32 bp, Artificial Sequence
  69-70WT-MB
cgcgatatgctatacatgtctctgggatcgcg

SEQ ID NO: 11 - DNA, 32 bp, Artificial Sequence
  69-70dd-MB
cgcgattgctatctctgggaccaatgatcgcg

SEQ ID NO: 12 - DNA, 22 bp, Artificial Sequence
  69-70F
caggacttgttcttacctttct

SEQ ID NO: 13 - DNA, 21 bp, Artificial Sequence
  69-70R
cagggttatcaaacctcttag